(19) United States
(12) Patent Application Publication
(10) Pub. No.: US 2017/0147752 A1
(43) Pub. Date: May 25, 2017

(54) Health data management device and health data management system

(71) Applicant: OMRON HEALTHCARE CO., LTD., Muko-shi (JP)

(72) Inventors: Toru DENO, Muko-shi (JP); Shusuke ESHITA, Muko-shi (JP); Yu MURATA, Muko-shi (JP); Yuka TANABE, Muko-shi (JP)

(73) Assignee: OMRON HEALTHCARE CO., LTD., Muko-shi (JP)

(21) Appl. No.: 15/244,752

(22) Filed: Aug. 23, 2016

(30) Foreign Application Priority Data
Jul. 3, 2015 (JP) 2015-134613

(51) Int. Cl. G06F 19/00; A61B 5/11; A61B 5/021; A61B 5/00; A61B 5/145

(52) U.S. Cl. CPC G06F 19/322; G06F 19/3481; A61B 5/742; A61B 5/14532; A61B 5/021; A61B 5/4869; A61B 5/11; A61B 5/0022; G06F 19/3418

(57) ABSTRACT

A health data management device includes a communication unit that receives index data based on a result of measurement from a measuring device for measuring biological information and activity information of a person to be measured, through wireless communication, a storage unit that stores a database for managing the received index data, a display unit that displays index data managed in the database, in a predetermined displaying way, and a control unit that controls an image displayed on the display unit. The control unit causes an index panel of each index data to be displayed on the display unit, for at least a part of the index data managed in the database. The index panel is an icon showing a value of each index data.

### BACKGROUND OF THE INVENTION

1. Technical Field

The present invention relates to a device and a system for managing indexes concerning health based on measured biological information (a body temperature, a blood pressure, and so on.) and activity information (the number of steps, and so on.) of an individual.

2. Related Art

A variety of healthcare systems have been developed to measure biological information (a pulse rate, a blood pressure, a body fat, and so on.) and activity information (the number of steps, a walking distance, and so on.) of an individual with measuring devices, register results of the measurement into a database, and manage the registered information (e.g., see Japanese Unexamined Patent Application Publication Nos. 2003-078644 and 2002-109064).

The healthcare systems of Japanese Unexamined Patent Application Publication Nos. 2003-078644 and 2002-109064 transmit information, such as a pulse rate, a blood pressure, a body fat, a weight, the number of steps, a walking distance, and the like, of an individual to a server on a network, and register the information on a database of the server. The healthcare systems transmit the data registered on the database from the server to a terminal of the individual, upon receipt of request for data from the terminal.

In the healthcare systems of Japanese Unexamined Patent Application Publication Nos. 2003-078644 and 2002-109064, data needs to be acquired from the server in order to use the data on the terminal side, and hence the terminal needs to be connected to the network. In an environment with a poor network connection, there may be problems in that the terminal could not be connected or the connection would take a long time, impairing user convenience.

Meanwhile, with the spread of smartphones, users have an increasingly strong desire to systematically manage a variety of information on their smartphones. This also applies to a healthcare application, and a healthcare application which is easily operable on a smartphone has been desired.

### SUMMARY OF THE INVENTION

The present invention provides a health data management device and a health data management system which enables easy use of index data showing a health condition of an individual and measured by a measuring device.

In a first aspect of the present invention, a health data management device is provided. The health data management device includes: a communication unit that receives index data based on a result of measurement from a measuring device for measuring biological information and activity information of a person to be measured, through wireless communication; a storage unit that stores database for managing the received index data; a display unit that displays index data managed in the database in a predetermined display method; and a control unit that controls an image displayed on the display unit. The control unit causes an index panel of each index data to be displayed on the display unit, for at least a part of the index data managed in the database. The index panel is an icon showing a value of each index data.

In this manner, the database is provided inside the health data management device, managing index data measured by the measuring device. Thus, in referring to data managed in the database, the health data management device does not need to be connected with a server over a network, so that the data in the database can be used regardless of a network connection environment. Since each of the index data managed in the database is displayed on the index panel on the display unit, the user can visually recognize the index data with ease. Accordingly, the present health data management device enables easy use of index data showing a health condition of an individual and measured by the measuring device.

In a second aspect of the present invention, a health data management system is provided. The health data management system includes: a measuring device that measures biological information and activity information of a person to be measured, and transmits index data based on a result of the measurement; and the health data management device that receives index data from the measuring device.

Similarly to the health data management device according to the first aspect, this health data management system also enables the user to easily use index data showing a health condition of an individual, measured by the measuring device.

In a third aspect of the present invention, a non-transitory recording medium storing a recorded program is provided. The program is a program that makes a computer of an information processor execute the functions of: receiving index data based on a result of measurement from a measuring device for measuring biological information and activity information of a person to be measured through wireless communication; registering the received index data on a database provided in the information processor; and displaying, on a display unit of the information processor, an index panel that is an icon showing a value of each index data, for at least a part of index data registered on the database.

With this program, a data management device can be realized to allow the user to easily use index data showing a health condition of the user (an individual), measured by a measuring device.

### BRIEF DESCRIPTION OF DRAWINGS

FIG. 1 is a diagram showing a mobile data terminal and a measuring device which are included in a health data management system according to an embodiment of the present invention;

FIG. 2 is a diagram for explaining a state of the mobile data terminal that receives data from a variety of measuring devices;

FIG. 3 is a view for explaining an example of a home screen of a healthcare application, displayed on a display unit of the mobile data terminal;

FIG. 4 is a view showing an example of a network configuration of the health data management system according to an embodiment of the present invention;

FIG. 5 is a diagram showing a configuration of a mobile data terminal;

FIG. 6A is a diagram showing a data structure of a device information table, and FIG. 6B is a diagram showing a data structure of a panel information table;

FIG. 7 is a diagram showing a data structure of an index database;

FIG. 8 is a diagram showing a configuration of a measuring device;

FIG. 9 is a flowchart showing control of display of a home screen upon start of the healthcare application;

FIGS. 10A and 10B are views for explaining examples of panels (examples of a blood pressure monitor and a blood glucose monitor) that vary in accordance with a type of a registered measuring device.

FIGS. 11A and 11B are views for explaining examples of panels (examples of a pedometer and a weight and body composition monitor) that vary in accordance with a type of a registered measuring device.

FIGS. 12A and 12B are views for explaining a state of change in chroma (saturation) of a background of the panel with a length of a non-updating period;

FIG. 13 is a flowchart showing control of changing chroma (saturation) of the background of the panel with the length of the non-updating period;

FIG. 14 is a flowchart showing control of background data transfer;

FIGS. 15A to 15D are views for explaining an operation for batch data transfer;

FIG. 16 is a flowchart showing control of the batch data transfer;

FIGS. 17A and 17B are views for explaining an operation for separate data transfer;

FIG. 18 is a flowchart showing control of separate data transfer:

FIGS. 19A to 19C are views for explaining an operation for panel movement;

FIG. 20 is a flowchart showing control of moving panel;

FIGS. 21A to 21D are views for explaining index charts displayed on the display unit of the mobile data terminal;

FIG. 22 is a flowchart showing control of display of an index chart.

FIGS. 23B to 23D are views for explaining another example of index charts displayed on the display unit of the mobile data terminal;

FIGS. 24A to 24C are views for explaining an operation for changing a display condition for an index chart (a data measurement period);

FIGS. 25A to 25C are views for explaining an operation for combining panels;

FIG. 26 is a flowchart showing control of combining panels; and

FIG. 27 is a view for explaining a combined index chart displayed on the combined panel.

### DETAILED DESCRIPTION OF PREFERRED EMBODIMENT

Hereinafter, an embodiment of the present invention will be described in detail with reference to the drawings.

### 1. Configuration

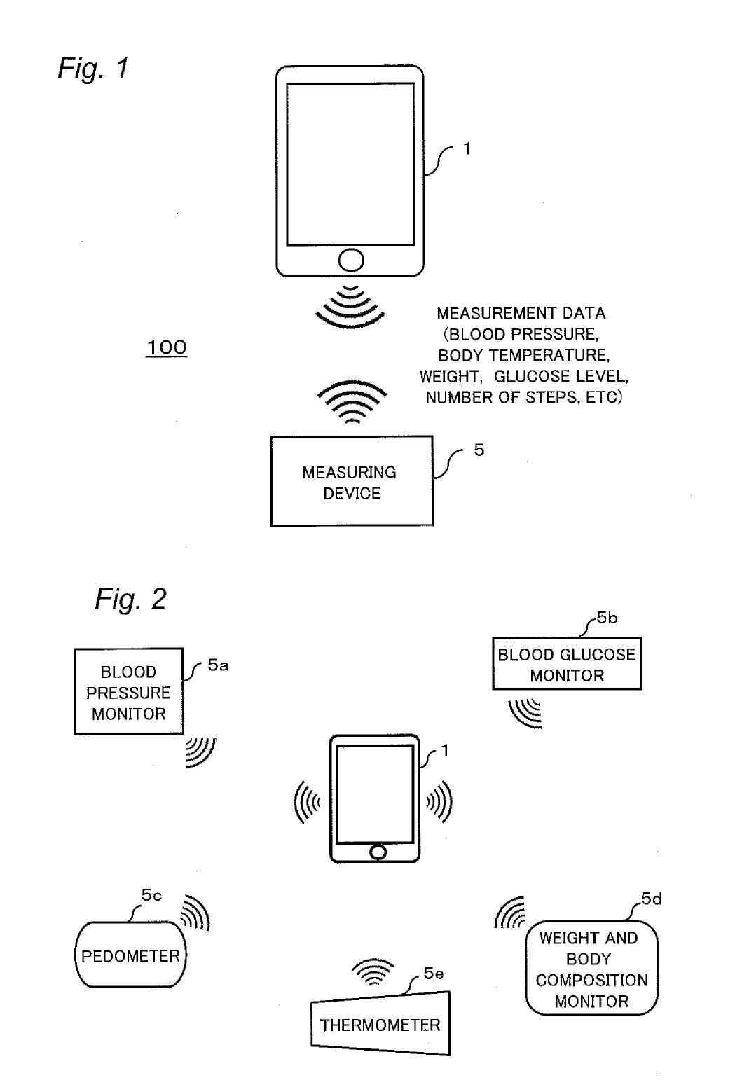

FIG. 1 is a diagram showing a configuration of a health data management system. A health data management system 100 includes a mobile data terminal 1 for managing index data, and a measuring device 5 for measuring index data.

The measuring device 5 is a device for measuring biological information, such as a body temperature, a blood pressure, a blood glucose level, a pulse rate, a body fat of a user (a person to be measured), and information concerning activities, such as the number of steps and a walking distance of the user. The measuring device 5 is, for example, a blood pressure monitor, a blood glucose monitor, a pedometer, a weight and body composition monitor, a thermometer, or an activity monitor. The measuring device 5 transmits measured values (a weight, a blood pressure, the number of steps, a BMI (body mass index), consumed calories and so on) to the mobile data terminal 1 as index data concerning health via wireless communication.

The mobile data terminal 1 registers the index data received from the measuring device 5 to an internal database. The mobile data terminal 1 is an information processing apparatus wirelessly communicable with another device and capable of storing data to perform a variety of data processing. The mobile data terminal 1 is, for example, a smartphone, a tablet terminal, a personal computer, or a wearable terminal. In the following description, the mobile data terminal 1 is assumed to be a smartphone as an example.

That is, as shown in FIG. 2, the mobile data terminal 1 is wirelessly communicable with a variety of measuring devices 5 such as a blood pressure monitor 5a, a blood glucose monitor 5b, a pedometer 5c, a weight and body composition monitor 5d, and a thermometer 5e. The mobile data terminal 1 has a function of acquiring index data (a blood pressure, a blood glucose level, the number of steps, or the like) from each of the measuring devices 5a to 5e to manage the data within the terminal.

Note that the mobile data terminal 1 acquires index data only from a measuring device 5 that is registered in advance. Hence the measuring device for transmitting index data to the mobile data terminal 1 needs to be registered in advance to a table (detailed later) which is managed by the mobile data terminal 1.

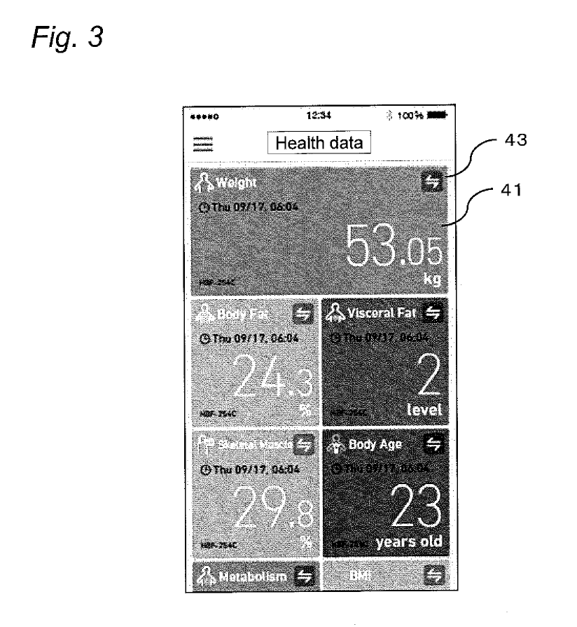

The mobile data terminal 1 registers the acquired index data on the database. The index data registered on the database can be displayed in a display method as shown in FIG. 3. The screen shown in FIG. 3 displays a plurality of icons (hereinafter referred to as “index panels”) 41 each showing the latest index value. FIG. 3 shows, as an example, the index panels indicating a weight, a body fat, a visceral fat, a skeletal muscle, a body age, and the like. The respective index panels are displayed with different colors so as to be easily viewable. Such panel display can facilitate the user to confirm a status concerning a health condition and activity of his/her self. Note that the screen shown in FIG. 3 is displayed when the user operates (taps) an icon indicating a healthcare application (detailed later) displayed on a display unit 13 of the mobile data terminal 1. Hereinafter, this screen is referred to as a “hone screen.”

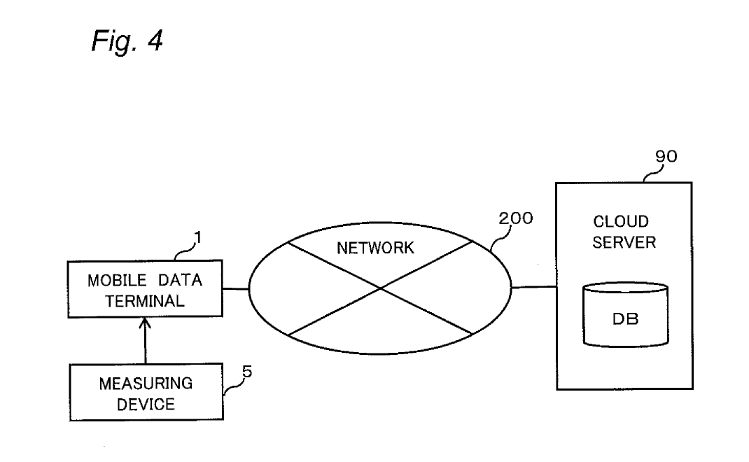

As shown in FIG. 4, the mobile data terminal 1 may be connected to a cloud server 90 over a network (e.g., an Internet) 200. The mobile data terminal 1 may transmit index data managed by itself to the cloud server 90 through the network 200 so that the index data is managed in the cloud server 90. Further, the mobile data terminal 1 may acquire index data managed in the cloud server 90 through the network 200 to use the acquired data.

1.1 Mobile Data Terminal

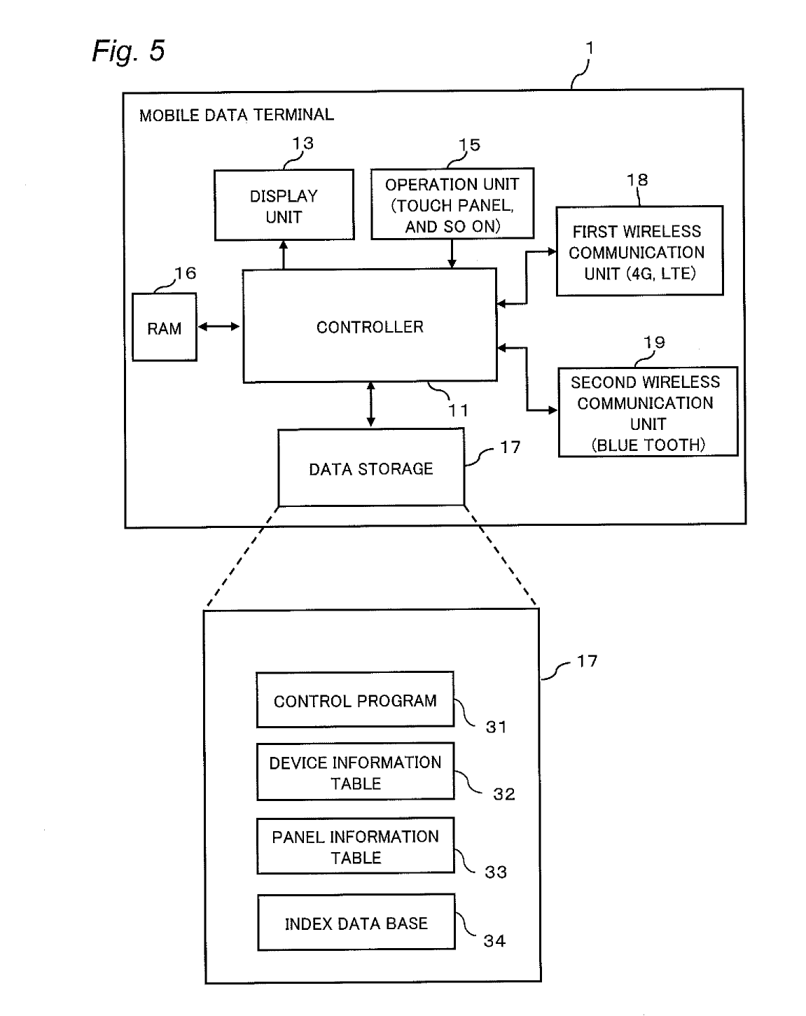

FIG. 5 is a diagram showing a configuration of the mobile data terminal 1. The mobile data terminal 1 includes a controller 11 for controlling the whole operation of the terminal, a display unit 13 for displaying a variety of information, an operation unit 15 that is operated by the user, a RAM 16, and a data storage unit 17 for storing data and a program. Further, the mobile data terminal 1 includes first and second wireless communication units 18 and 19 for communicating with an external apparatus.

The display unit 13 is configured by a liquid crystal display device or an organic EL (electroluminescence) display device, for example. The operation unit 15 includes a touch panel and a button. The touch panel is disposed such that its operation region is overlapped on a display region of the display unit 13. The button includes a button physically provided in the mobile data terminal 1, and a button virtually displayed on the display unit 13.

The first wireless communication unit 18 is a circuit (module) for performing communication in accordance with a communication standard such as 3G, 4G, LTE, or WiMAX (registered trademark). The second wireless communication unit 19 is a circuit (a module) for performing communication in accordance with a Bluetooth (registered trademark) standard.

The RAM 16 is configured by a semiconductor device such as a DRAM or an SRAM, temporarily storing data and functioning as operation area of the controller 11.

The data storage unit 17 is a recording medium for storing parameters required for realizing a predetermined function, data, control programs, and the like. The data storage unit 17 is configured by a hard disk (HDD) or a semiconductor storage device (SSD), for example.

The controller 11 executes a control program 31 (software) to realize a predetermined function. In the present embodiment, a healthcare application program (hereinafter referred to as “healthcare app”) is previously stored in the data storage unit 17 as a control program, and the controller 11 executes the healthcare app to realize functions described below.

1.2 Table and Database

As shown in FIG. 5, the data storage unit 17 stores, in addition to the control program 31 to be executed by the controller 11, a device information table 32, a panel information table 33, and an index database (DE) 34 as information required for controlling the apparatus. Each of tables and database will be described below.

(1) Device Information Table

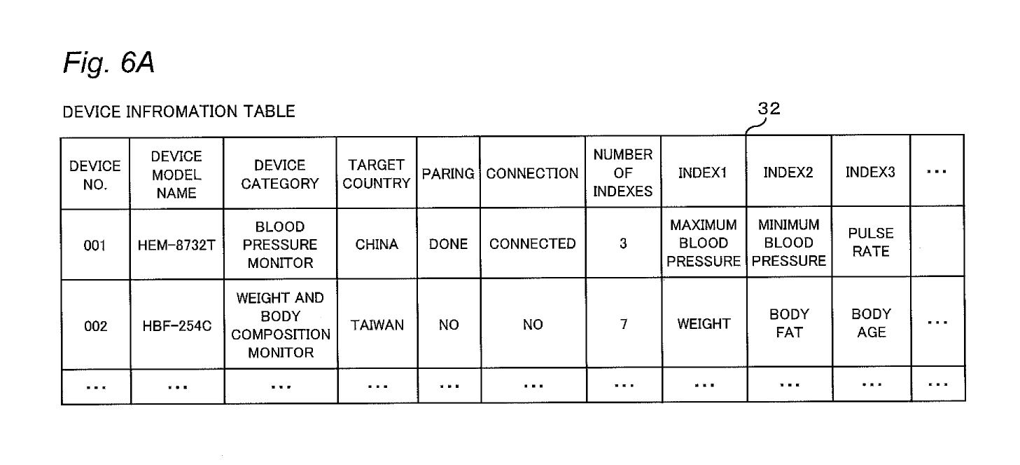

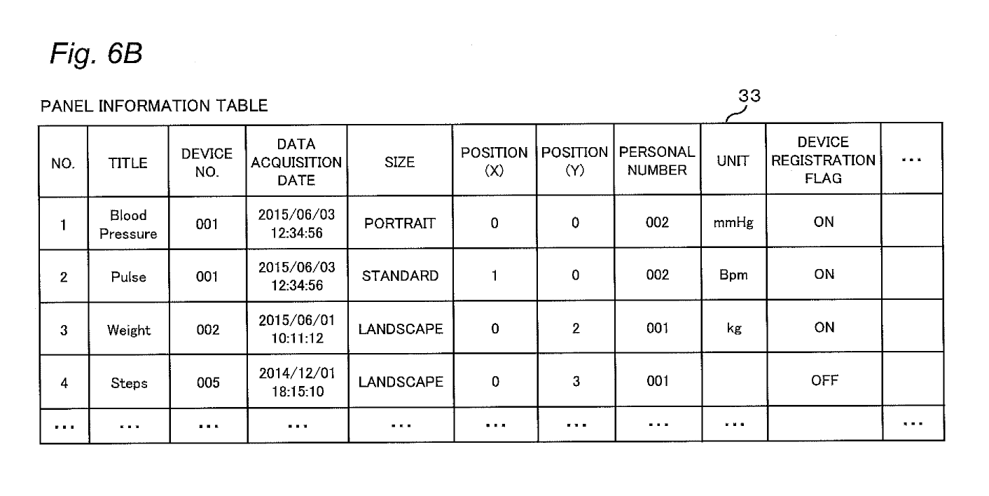

The device information table 32 is a table for managing the measuring devices 5 which are registered in the mobile data terminal 1. That is, information (device model name, device category, and so on) of the measuring device 5 is registered on the device information table 32 so that the measuring device 5 is registered in the mobile data terminal 1. FIG. 6A shows a data structure of the device information table 32. The device information table 32 has items of device model name, device category, target country, pairing, connection, number of indexes, Index 1, Index 2, Index 3, . . . .

“device model name” stores information showing a model name of the measuring device 5. “device category” stores information showing a type of the measuring device 5. “target country” stores information showing a country in which the present healthcare app is being used. “paring” stores information showing whether or not paring between the mobile data terminal 1 and the measuring device 5 have been established. “connection” stores information showing whether or not communication has been established between the mobile data terminal 1 and the measuring device 5.

Index 1, Index 2, Index 3, . . . store index values acquired from the measuring device 5. Specifically, indexes to be allocated to the measuring device vary depending on type of the measuring device. For example, when the measuring device 5 is the blood pressure monitor, “maximum blood pressure”, “minimum blood pressure”, “pulse rate”, and the like are respectively allocated to Index 1, Index 2, Index 3, . . . . When the measuring device 5 is the weight and body composition monitor, “weight”, “body fat”, “body age”, and the like are respectively allocated to Index 1, Index 2, Index 3, . . . .

(2) Panel Information Table

The panel information table 33 is a table for managing information about the index panel 41 (cf. FIG. 3) displayed on the display unit 13 of the mobile data terminal 1. That is, only an index panel registered on the panel information table 33 is displayed on the screen that is displayed when an icon of the healthcare app is operated on the mobile data terminal 1. FIG. 6B shows a data structure of the panel information table 33.

The panel information table 33 includes items of title, device No., data acquisition date, size, position (X), position (Y), personal number, unit, device registration flag, and the like. Title is a name of the index panel. For example, a panel showing a blood pressure measurement result is named “Blood Pressure.” Device No. is a number (an identification number) for identifying the measuring device 5. By referring to the device information table 32 with the device No., the device model name of the measuring device 5 associated with each of index panel and the index name can be grasped.

“Data acquisition date” shows the latest date of acquiring the index concerning the index panel. “Size” shows a size of the index panel. “Position (X)” and “Position (Y)” show a displayed position (coordinates) of the index panel on the display unit 13. “Personal number” is a number for identifying the user. “Unit” is a unit of a measured value displayed on the index panel. “Device registration flag is a flag showing whether or not the measuring device 5 corresponding to the index panel is currently registered in the mobile data terminal 1. The device registration flag being “ON” indicates that the measuring device corresponding to the index panel is currently registered in the terminal, while the device registration flag being “OFF” indicates that the measuring device has been deleted after registration.

(3) Index Database

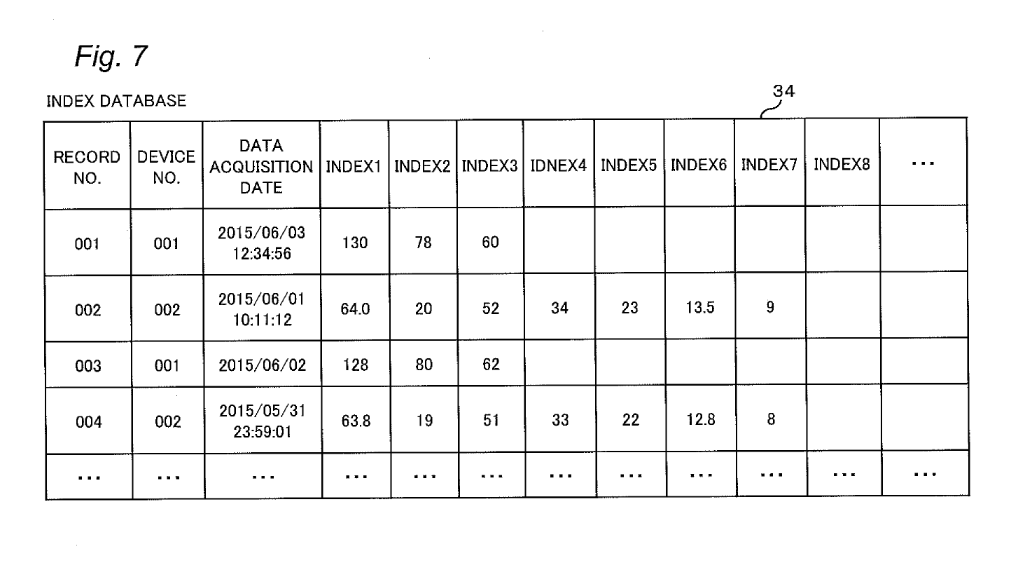

The index database 34 is a database for managing index data received from each measuring device 5. FIG. 7 shows a data structure of the index database 34. The index database 34 manages data received from each measuring device 5 as one record. Specifically, the index database 34 manages items of record No., device No., data acquisition date, Index 1, Index 2, Index 3, . . . . “Record No.” is a number of a record. “Device No.” is a number (an identification number) for identifying the measuring device 5. “Data acquisition date” is a date of acquiring data from the measuring device 5. Indexes 1, 2, . . . store index values acquired from the measuring device 5. Specifically what indexes are allocated vary depending on the measuring device 5.

1.3 Measuring Device

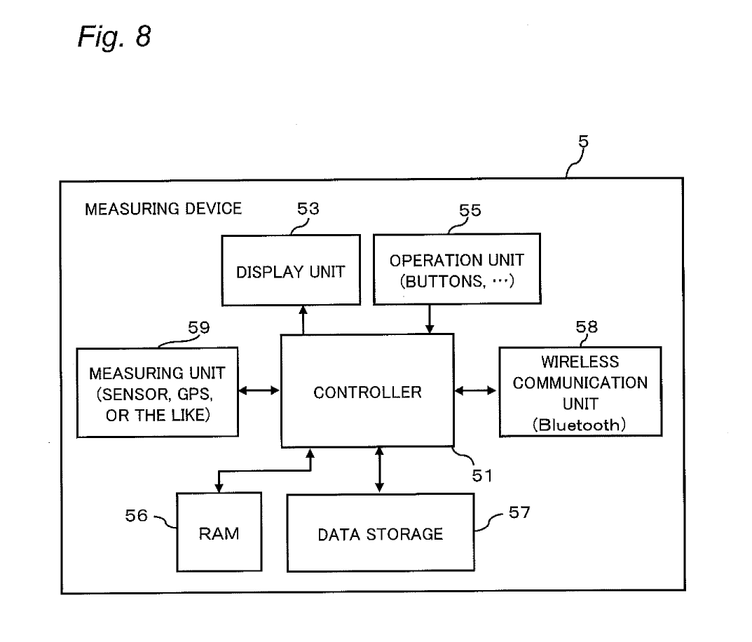

FIG. 8 shows a configuration of the measuring device 5. The measuring device 5 includes a controller 51 for controlling the whole operation of the device, a display unit 53 for displaying a variety of information, an operation unit 55 that is operated by the user, a RAM 56 for temporarily storing data, a data storage unit 57 for storing data and programs, a wireless communication unit 58 for communicating with an external apparatus (particularly, the mobile data terminal 1), and a measurement unit 59 for measuring biological information and the like.

The display unit 53 is configured by a liquid crystal display device or an organic EL (electroluminescence) display device, for example. The operation unit 55 is a user interface for receiving user's operation, such as a button and a touch panel. The button includes a button physically provided on the measuring device 5 and a virtual button displayed on the display unit 13.

The wireless communication unit 58 is a circuit (module) for communication in accordance with the Bluetooth (registered trademark) standard.

The RAM 56 is configured by a semiconductor device such as a DRAM or an SRAM, temporarily storing data and functioning as an operation area of the controller 51.

The data storage unit 57 is a recording medium for storing parameters required to realize a predetermined function, control programs, measured value of the index, calculated index value, and the like. The data storage unit 57 is configured by a hard disk (HDD) or a semiconductor storage device (SSD), for example.

The controller 51 executes a control program to realize a predetermined function. In the present embodiment, a healthcare application program (healthcare app) for a measuring device is previously stored in the data storage unit 57 as a control program, and the controller 51 executes the healthcare app to realize a function described below.

The measurement unit 59 includes a sensor for measuring a variety of biological information such as a blood pressure, a blood glucose, a body temperature, a pulse rate, and the number of steps, and a sensor for measuring a current position, such as a GPS (global positioning system). The types of sensors to be included in the measurement unit 59 varies depending on the function of the measuring device 5, namely the measurement target.

The measuring device 5 transfers information measured by the measurement unit 59 as index data to the mobile data terminal 1 through the wireless communication unit 58, the measured information including blood pressure, blood glucose, body temperature, pulse rate, the number of steps, walking distance, BMI, consumed calories, and/or the like. Note that the information measured by the measuring device 5 as a measurement result includes measured values (a body temperature, a blood pressure, a blood glucose level, and the like) measured by the measurement unit 59, and calculated values (a BMI, consumed calories, and the like) each calculated in view of a measured value and another element.

### 2. Function and Operation

Operation of the health data management system 100 configured as above will be described below. A function and operation described below are realized by the controller 11 of the mobile data terminal 1 executing a program of the healthcare app.

As shown in FIG. 2, the mobile data terminal 1 receives index data from each of measuring devices 5a, 5b, . . . , and stores the received data in the index database 34. Since storing the index database 34 therein, the mobile data terminal 1 can refer to the index database 34 at any time to acquire index data without connection to the external network.

2.1 Display of Home Screen

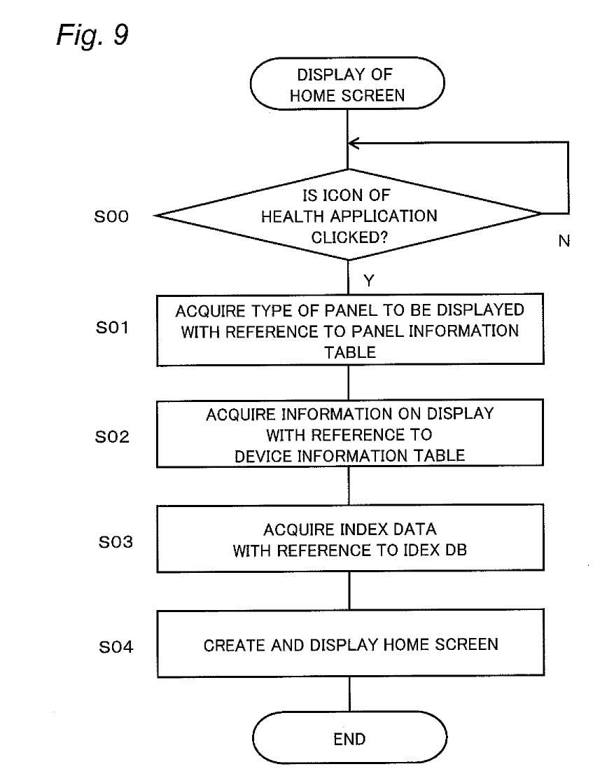

When the user clicks an icon (not shown) of the healthcare app displayed on the display unit 13 of the mobile data terminal 1, a home screen including panels each showing a value of each index is displayed, as shown in FIG. 3. With reference to a flowchart of FIG. 9, control of displaying the home screen by the healthcare app will be described.

When the icon of the healthcare app on the display unit 13 is clicked by the user (YES in S00), the controller 11 acquires information about the type of panel to be displayed on the display unit 13 by referring to the panel information table 33 (S01). Next, by referring to the device information table 32 with device No. registered in the panel information table 33 as a key, the controller 11 acquires a device model name and names of Indexes 1, 2, . . . for each device No. (S02). Further, the controller 11 searches the index database 34 with each of device Nos. registered in the panel information table 33, to acquire the latest value (namely, having the latest Data acquisition date) of each index for each device (S03). The controller 11 then creates a home screen including a panel on the basis of the acquired names and index values and displays the created home screen on the display unit 13 (S04). In this manner, the home screen as shown in FIG. 3 is displayed, including the index panel 41 that shows each of index.

2.2 Size of Index Panel

Regarding sizes of a plurality of index panels displayed on each measuring device 5, an index panel that shows an index representing the measuring device 5 is displayed in a larger size than the other index panels. For example, when the measuring device 5 is a blood pressure monitor, the size of the index panel showing the maximum and/or minimum blood pressures is made largest, and the other indexes (e.g., a pulse rate) are made smaller than the above panel. When the measuring device 5 is a weight scale, the index panel showing a weight is displayed in the largest size. That is, for each measuring device, a size of an index panel showing the most important index of the measuring device is made larger, and sizes of index panels showing the other indexes are made smaller.

When the number of index panels displayed on the display unit 13 becomes large, all the index panels may be made viewable by screen scroll, or a size of each index panel may be adjusted such that all the panels are held within one screen.

2.3 Addition of Index Panel

The measuring device 5 can be registered in the mobile data terminal 1 through a predetermined registration screen (not shown) displayed on the mobile data terminal 1. The registration of the measuring device 5 allows information of the device to be registered in the device information table 32. Upon the registration of a new measuring device 5 in the mobile data terminal 1 (i.e., the device information table 32), an index panel corresponding to the measuring device is added to the home screen (i.e., the panel information table 33).

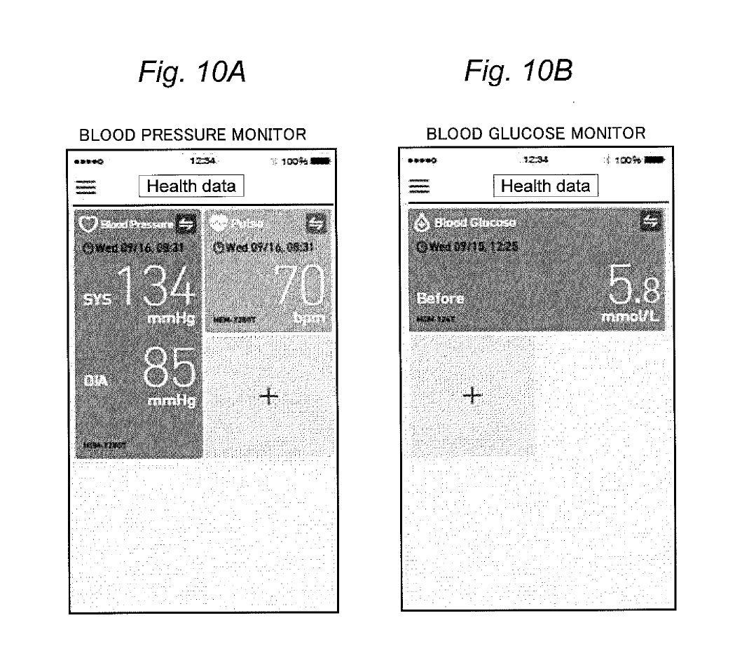

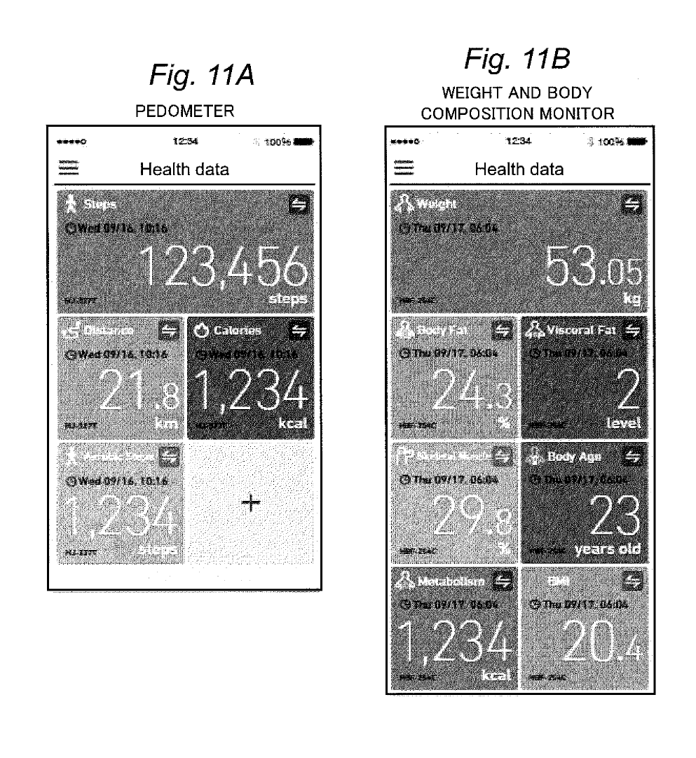

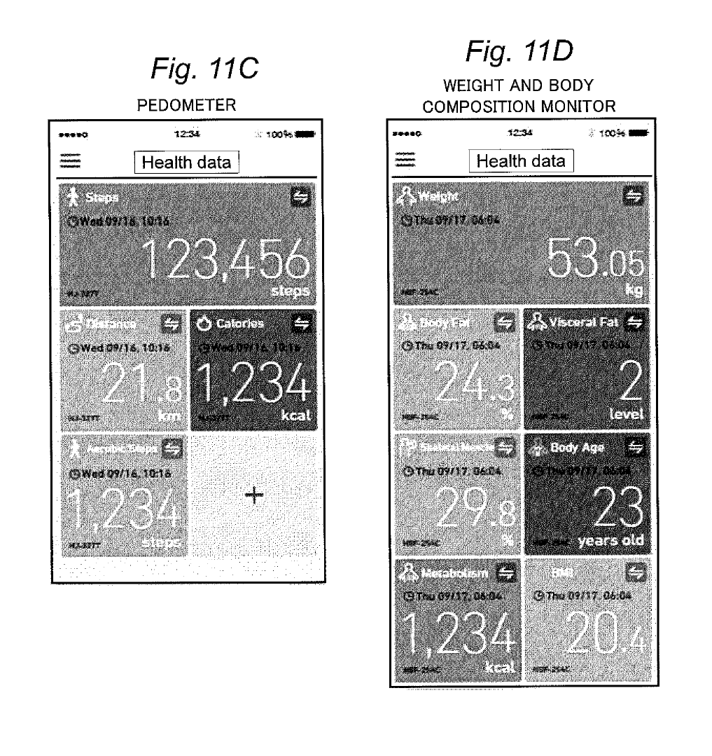

At this time, the added index panel is different for each measuring device. For example, when the measuring device 5 is the blood pressure monitor, index panels showing a blood pressure and a pulse rate, respectively, are added, as shown in FIG. 10A. When the measuring device 5 is the blood glucose monitor, an index panel showing a blood glucose level is added, as shown in FIG. 10B. When the measuring device 5 is the pedometer, index panels showing the number of steps, a walking distance, consumed calories, and the like, respectively, are added, as shown in FIG. 11A. When the measuring device 5 is the weight and body composition monitor, index panels showing a weight, a body fat, a visceral fat, and the like, respectively, are added, as shown in FIG. 11B.

A plurality of measuring devices in the same category (e.g., category of blood pressure monitor) can be registered in a single mobile data terminal 1. In this case, index panels of the respective measuring devices are added and the latest measurement results of the respective measuring devices are displayed. For example, it is possible to separately register an upper-arm blood pressure monitor suitable for installation at a home and a wrist blood pressure monitor easily carried for a business trip and a working place. In this case, index panels of the respective blood pressure monitors are added and the latest measurement results of the respective blood pressure monitors are displayed.

2.4 Change in Background Color of Index Panel

When the index shown by the index panel has not been updated for a long time, the healthcare app of the present embodiment changes a background color (chroma) of the index panel in accordance with the length of a period in which the index data has not been updated (hereinafter referred to as a “non-updating period), to inform the user of the non-updating. Specifically, as the non-updating period becomes longer, the chroma (saturation) of the background color of the index panel is changed so as to fade the background color. That is, as shown in FIGS. 12A and 12B, as non-updating periods for index data of index panels 41a and 41b become longer, background colors of the index panels 41a and 41b are changed to be faded from FIG. 12A to FIG. 12B. A color of an index panel 41c is not changed because its index data is relatively new. This color change facilitates the user to understand that the indexes of the index panels 41a and 41b among a plurality of index panels have not been updated for a long time.

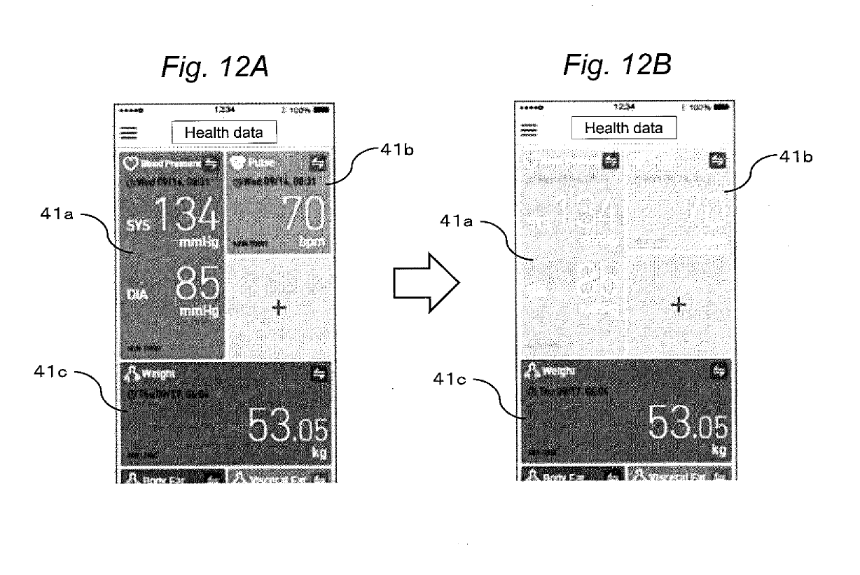

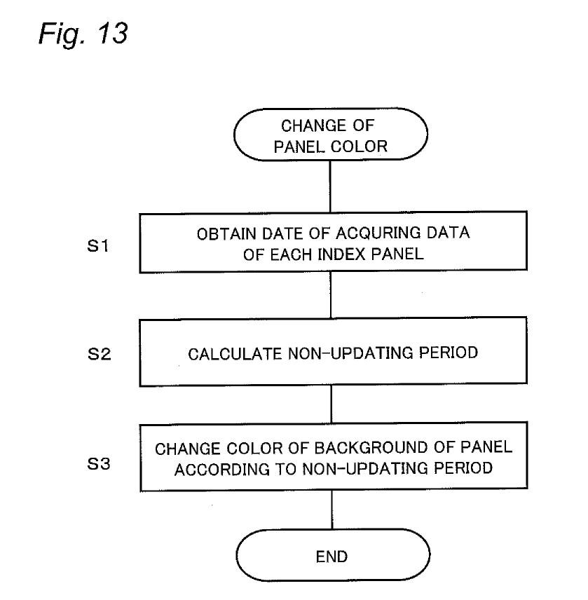

FIG. 13 is a flowchart showing control of change in display color of the index panel. The controller 11 refers to the panel information table 33 to acquire a data acquisition date concerning each index panel (S1). The controller 11 calculates a period in which data has not been updated for each index panel based on information of the acquired data acquisition date and the current date (S2). The controller 11 changes the background color of each index panel in accordance with the calculated length of the non-updating period for each index panel (S3). At this time, the background color is changed in a stepwise or continuous manner in accordance with the length of the non-updating period. This allows the user to intuitively grasp the length of the period in which the index data has not been updated.

2.5 Data Transfer (Update) in Back Ground

When the healthcare app appears not to be executed (with the home screen not displayed) but is executed in the back ground, the mobile data terminal 1 automatically acquires the latest index data periodically from the measuring devices. This eliminates necessity for the user to be particularly conscious of the updating timing, enabling the user to view the latest data at all times. Setting as to whether to transfer data in the back ground can be set by the user. Hereinafter, such a setting is referred to as “back ground transfer setting.”

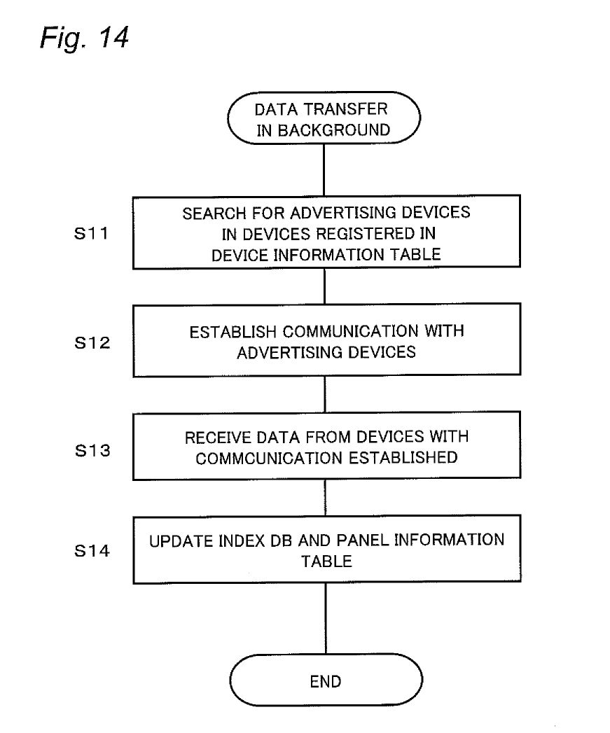

FIG. 14 is a flowchart showing control concerning data transfer in the back ground described as above. This control is executed periodically, namely every predetermined period, when the back ground transfer setting is on. The controller 11 refers to the device information table 32 to search for the measuring device 5 which is advertising in the measuring devices registered in the device information table 32 (S11).

Here, advertising is operation defined by the Bluetooth standard, and operation for notifying the presence of oneself to other devices. The measuring device 5 advertises itself at predetermined timing defined for each device type. For example, the measuring device 5 advertises itself during a predetermined period (e.g., three minutes) after pressing of a predetermined button, or performs advertising when there is data which has not been transmitted. Alternatively, the measuring device 5 may advertise itself for a predetermined period (e.g., 60 minutes) after measuring data of a measurement target.

The controller 11 establishes communication with the measuring device 5 which is advertising itself (S12). The controller 11 requests the measuring device 5 with which the communication has been established to transfer the latest index data to receive the latest index data (S13). The controller 11 updates the index database 34 with the received index data (S14).

As described above, when the back ground transfer setting is on, data is transferred in the back ground.

2.6 Batch Data Transfer (Update)

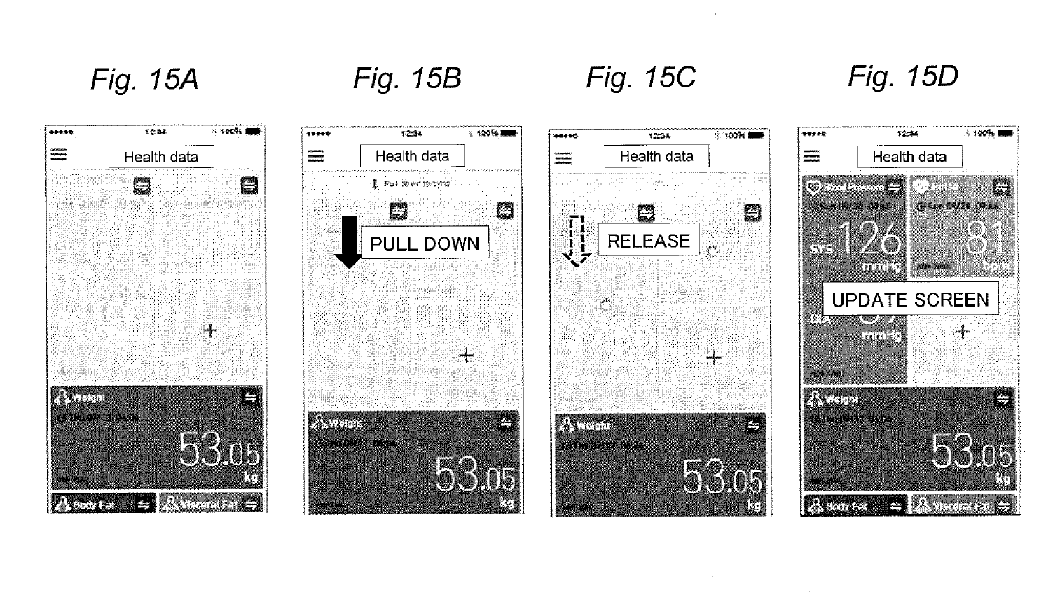

When the user performs operation of pulling down the home screen on the operation unit (touch panel) 15, the mobile data terminal 1 communicates with the registered measuring devices 5 to acquire index data in a batch from the measuring devices 5. Specifically, the user touches a certain region on the home screen as shown in FIG. 15A with his or her finger, slides down the region as shown in FIG. 15B, and then takes the finger off the screen (FIG. 15C). With such user's operation, the mobile data terminal 1 starts to communicate with the measuring devices 5 and acquires index data in a batch from the measuring devices 5. Then, the index database 34 is updated by the acquired index data, and the display of the index panels on the screen is also updated (FIG. 15D).

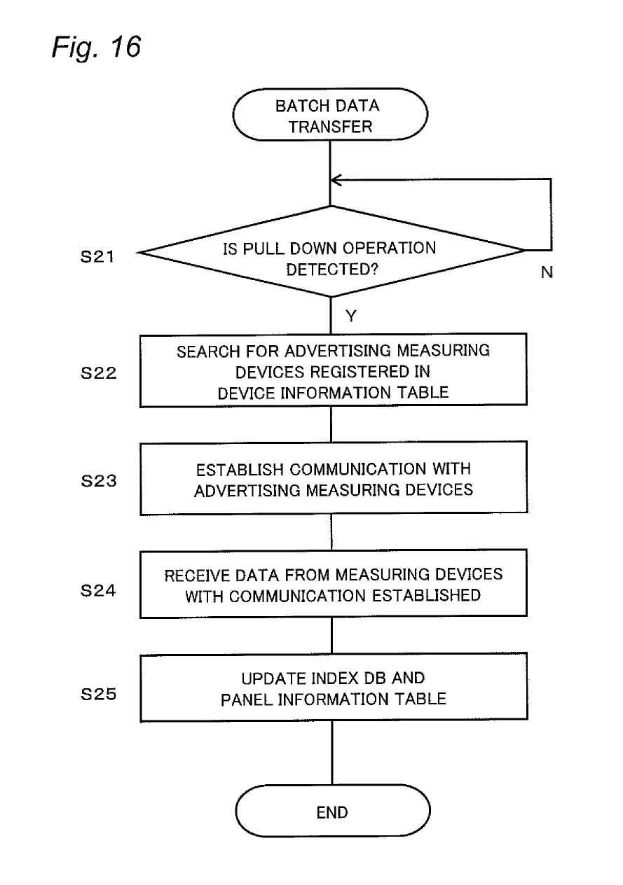

With reference to a flowchart of FIG. 16, the control regarding such batch data transfer will be described. The controller 11 determines whether or not the pull-down operation on the home screen is detected (S21). When the pull-down operation on the home screen is detected, the controller 11 searches for devices which are advertising among the measuring devices 5 registered in the device information table 32 (S22) and establishes communication with the measuring devices 5 which are advertising (S23). The controller 11 requests the measuring devices 5 with which the communication is established to transfer the latest index data, and receives index data from the measuring devices (S24). The controller 11 updates the index database 34 with the received index data (S25).

As described above, data is transferred when the user performs the pull-down operation on the home screen.

2.7 Separate Data Transfer

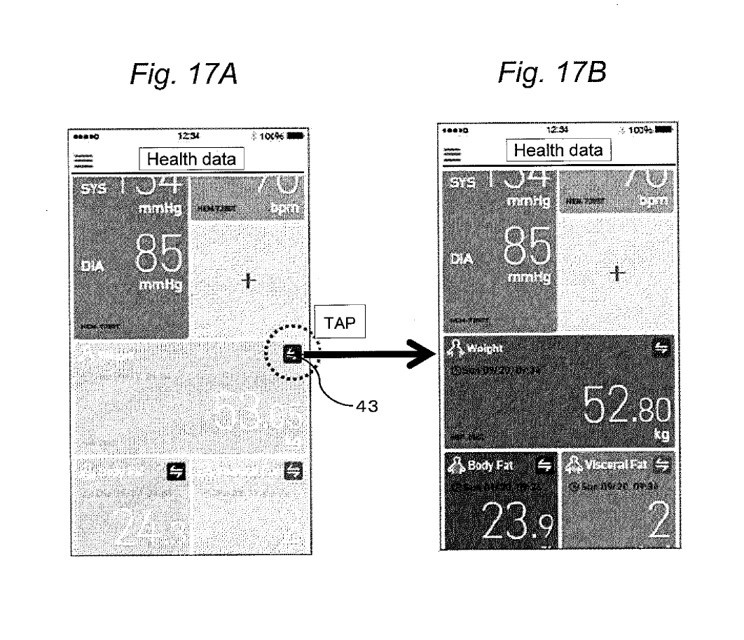

The healthcare app has a function of updating index data for each measuring device. For this purpose, an update button 43 is displayed in an upper right region of the index panel as shown in FIGS. 17A and 17B. When the update button 43 is tapped, index data is received from the measuring device associated with the index panel including the update button 43, and the index data is updated (FIG. 173). When the update button 43 is thus tapped, data is separately transferred from each measuring device, and the data is updated.

With reference to a flowchart of FIG. 18, the control concerning such separate data transfer (update) of the device will be described. The controller 11 detects whether or not the update button 43 is tapped (S31). When the update button 43 is tapped, the controller 11 refers to the panel information table 33 to identify the measuring device associated with the index panel of which update button 43 is tapped (S32). Then, the controller 11 searches for a device which is advertising through the second wireless communication unit 19, and determines whether or not the identified measuring device is detected among the advertising devices (S33). When the identified device is not detected (NO in S33), the present processing is finished.

When the identified device is detected (YES in S33), the controller 11 establishes communication with the detected measuring device through the second wireless communication unit 19 (S34). After establishing communication, the controller 11 requests the measuring device with which communication is established to transfer data and acquires index data therefrom (S35). On the basis of the acquired index data, the controller 11 updates the index database 34 and the panel information table (a data acquisition date) (S36). The controller 11 then changes the display of the index panel on the basis of the latest information (S37). Specifically, the index value and/or the background color on the index panel are/is changed.

As described above, data transfer (data update) can be performed with respect to each measuring device. In the above example, data transfer with respect to each measuring device is started by the tap operation on the update button provided on the index panel. However, data transfer with respect to each measuring device may be started by another operation on the index panel (e.g., tap operation on a region of the index panel).

2.8 Panel Move

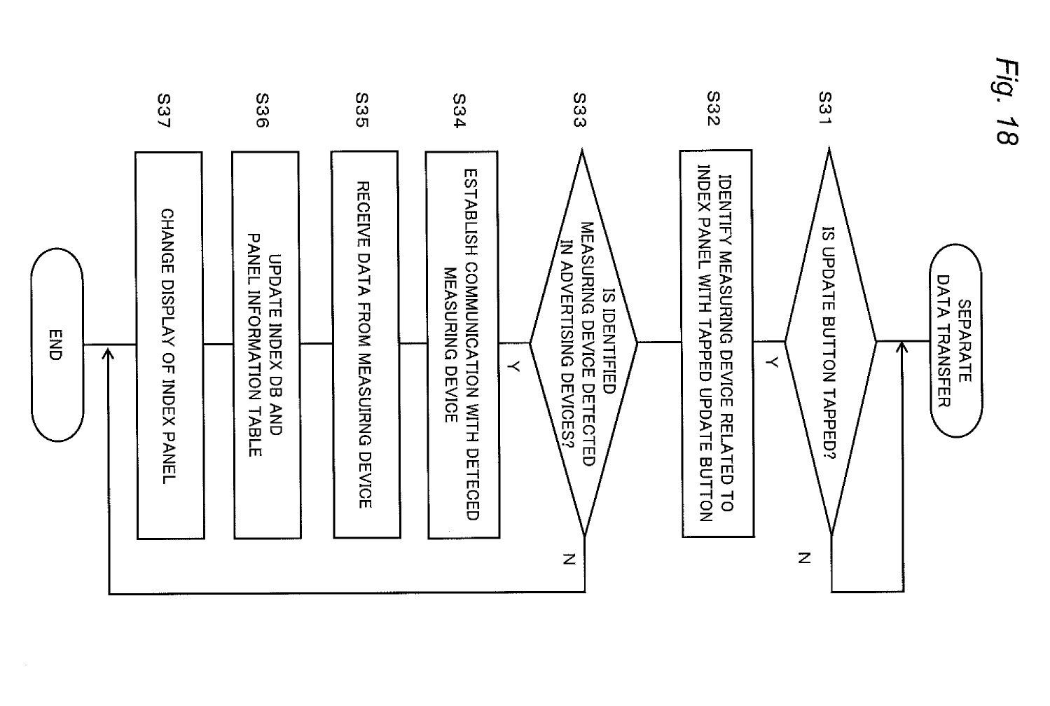

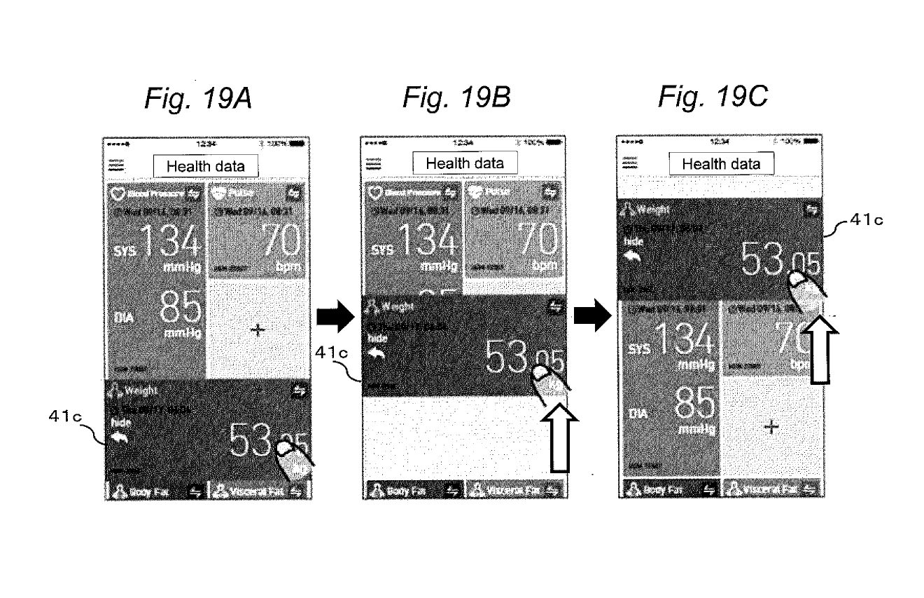

The healthcare app has a function of changing the position of the index panel on the home screen by user's operation. For example, as shown in FIGS. 19A to 19C, a drag operation on the index panel 41c moves it to a desired position. In this way, the position of the index panel 41c can be changed.

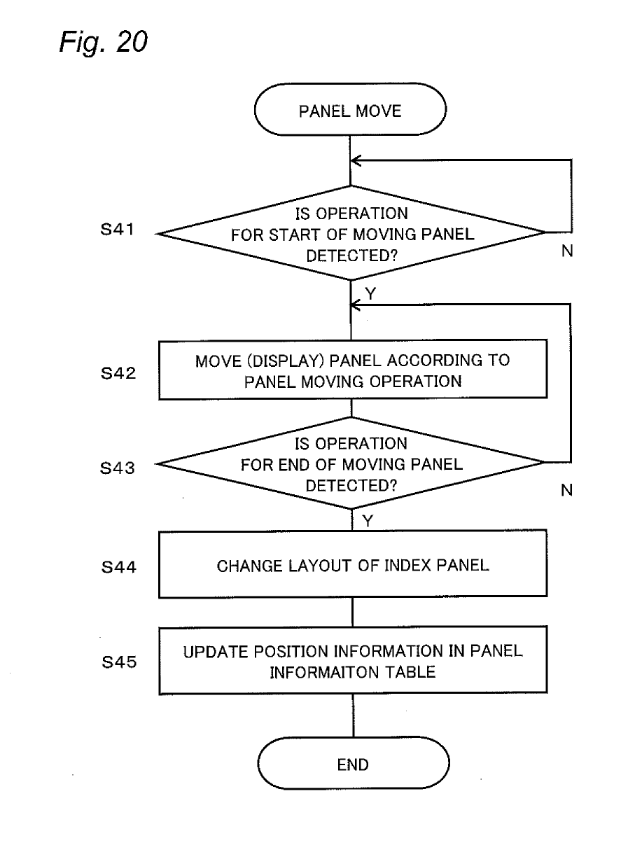

With reference to a flowchart of FIG. 20, the control of moving the index panel will be described. Upon detection of moving operation (drag operation) on the index panel (YES in S41), the controller 11 moves the index panel on the screen in accordance with the moving operation (see FIG. 19B) (S42). Subsequently, upon detection of end of the moving operation (YES in S43), the controller 11 changes a layout of the index panels so as to dispose the index panel moved in the moving operation on the position to which the index panel is moved in the moving operation (S44). The controller 11 then updates the panel information table 33 (S45). Specifically, values of the position (X) and the position (Y) of the index panel of which position is changed are updated in the panel information table 33.

As described above, the index panel can be moved, and the user can customize the layout to his or her preference.

When operation is performed so as to move an index panel out of the screen (the display region of the display unit 13), the index panel may be erased. That is, when an index panel is moved such that a portion being a predetermined percentage or more of the index panel protrudes out of the screen, the index panel may be erased.

Alternatively, the index panel may be automatically moved such that a frequently used index panel is disposed on the top of the screen. For this purpose, the controller 11 counts the number of taps on each index panel and stores the counted value in the data storage unit 17. The controller 11 periodically checks the counted value, and relocates each index panel on the basis of the counted value such that the frequently used panel is disposed on the upper side.

2.9 Graphical Display

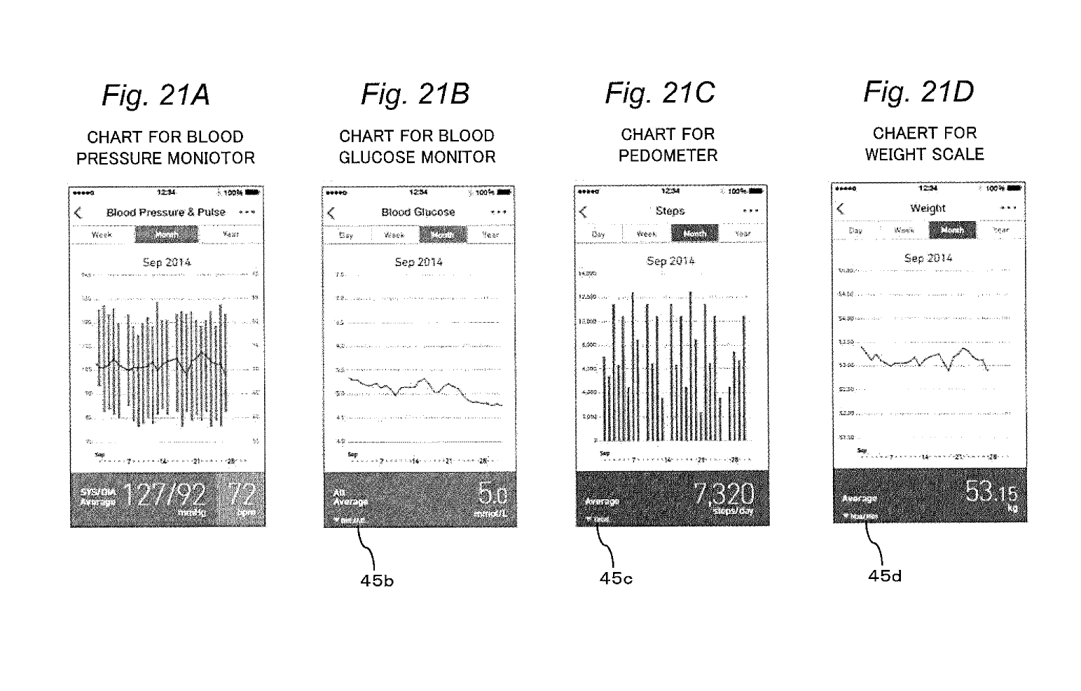

The healthcare app has a function of displaying a chart of an index indicated by an index panel on the home screen when the user taps a region of the index panel. FIGS. 21A to 21D are views showing charts that is displayed when a variety of index panels are tapped. FIG. 21A shows a chart for the blood pressure monitor which is displayed when the index panel showing a blood pressure is tapped. FIG. 21B shows a chart for the blood glucose monitor which is displayed when the index panel showing a blood glucose is tapped. FIG. 21C shows a chart for the pedometer which is displayed when the index panel showing the number of steps is tapped. FIG. 21D shows a chart for the scale which is displayed when the index panel showing a weight is tapped.

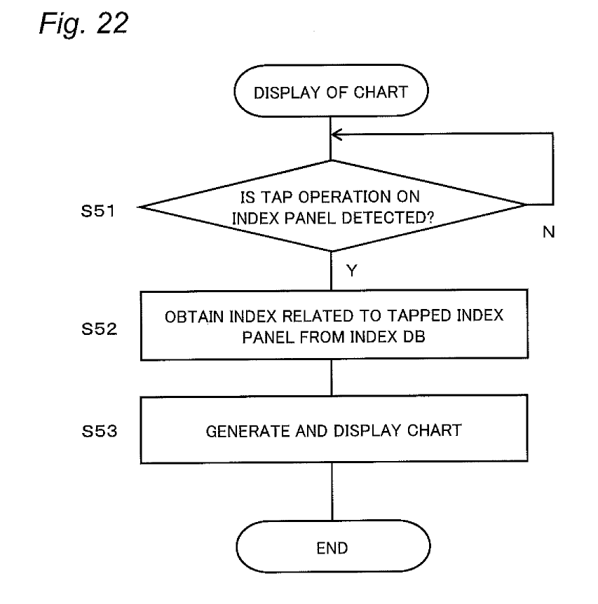

With reference to a flowchart of FIG. 22, the control of graphical display of an index will be described. Upon detection of tap operation on the index panel (YES in S51), the controller 11 acquires data to be used in a chart from the index database 34 regarding an index associated with the tapped index panel (S52), generates a chart on the basis of the acquired data, and displays it on the display unit 13 (S53).

As described above, each index can be displayed graphically. In the above example, graphical display is performed by the tap operation on the region of the index panel. However, graphical display may be performed by another operation on the index panel (e.g., operation on a button provided on the index panel).

Although a horizontal axis of each chart shows a period, that period can be switched to a week (Week), a month (Month), or a year (Year). With the period optionally settable, the user can grasp a temporal variation in each index for a desired period (a daily variation, a variation per day of the week, a mid-term variation, a long-term variation, or the like).

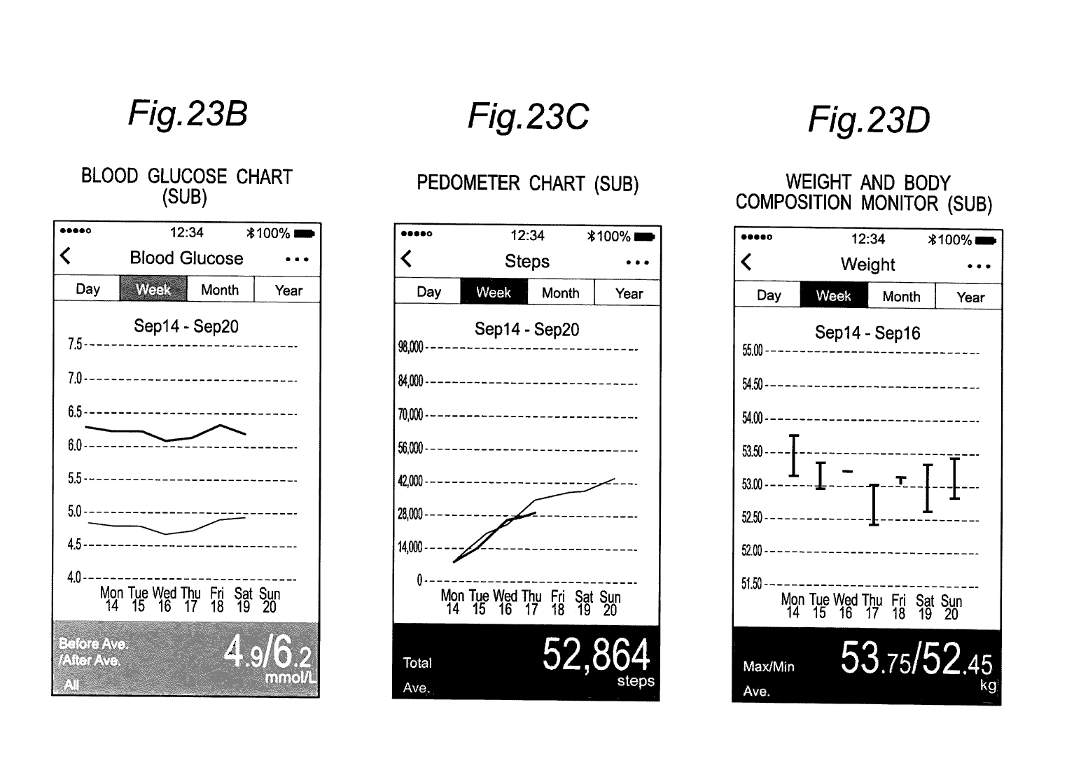

The index can also be displayed graphically from a variety of viewpoints. Hence buttons 45b to 45d are prepared on the graphical display screens as shown in FIGS. 21A to 21D. For example, regarding the blood glucose level, tapping the button 45b in the chart as shown in FIG. 21B can lead to further display of a chart showing a state of change in average values of the blood glucose levels respectively before and after eating as shown in FIG. 23B. Regarding the number of steps, tapping the button 45c in the chart shown in FIG. 21C can lead to display of a chart comparing progress in activity between a previous period and the present as shown in FIG. 23C. Regarding the weight, tapping the button 45d in the chart shown in FIG. 21D can lead to display of the maximum value and the minimum value of the weight in a day as shown in FIG. 23D. The user can thereby see a variation in weight in a day.

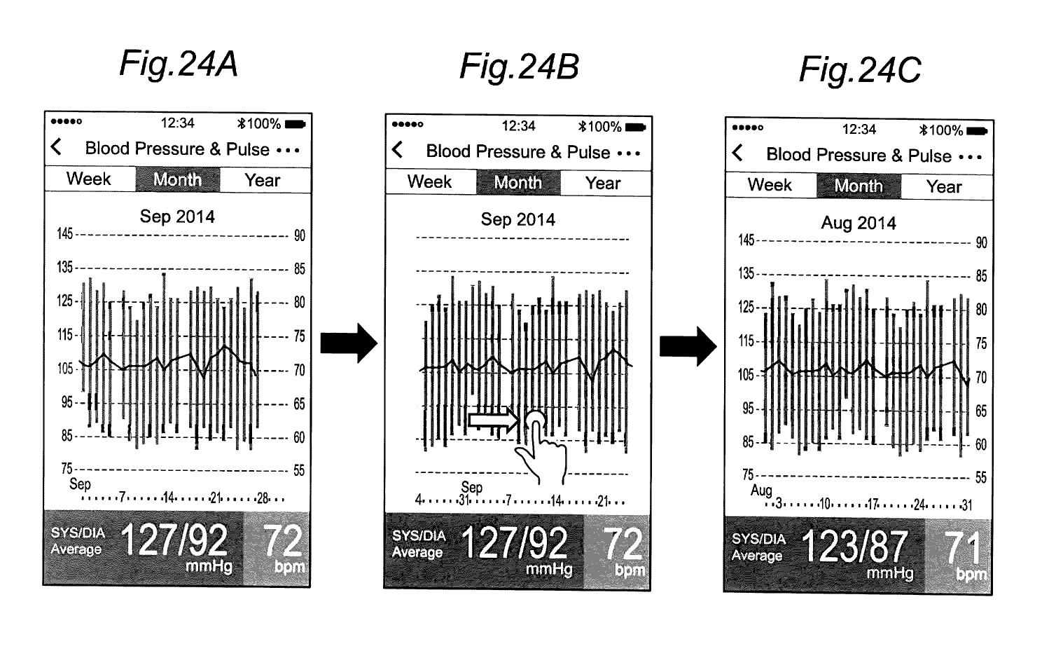

When slide operation in a horizontal direction (to a previous or subsequent period) is performed on the chart displayed on the display unit 13, the chart is displayed with its display period switched to a previous or subsequent display period. For example, when the slide operation is performed to the right, a chart for data in the previous period is displayed. For example, in the case of FIGS. 24A to 24C, when slide operation is performed to the right as shown in FIG. 243 in an initial state where data of September, 2014 is graphically displayed as shown in FIG. 24A, data of August, 2014 which is a previous period is graphically displayed as shown in FIG. 24C.

When a plurality of measuring devices in the same category are registered in the mobile data terminal 1, a chart displayed from the index panel of each of the measuring devices shows a value obtained by merging a value of each of the measuring devices in the same category. That is, when displaying a chart concerning an index of one measuring device, the controller 11 merges a measurement result of the measuring device and a measurement result of another measuring device in the same category, to generate the chart. For example, when an upper-atm blood pressure monitor which is suitable for placement in a home and a wrist blood pressure monitor which is easily carried for a business trip and a working place are registered in the mobile data terminal 1, as a chart displayed from the index panel of the blood pressure monitors, a chart generated by merging a measurement result of the upper-arm blood pressure monitor and a measurement result of the wrist blood pressure monitor is displayed. Hence it is possible to grasp in a batch a blood pressure value measured in an individual residence (e.g., values measured in the morning and evening) and a blood pressure value measured in a working place (e.g., a value measured in the daytime).

As described above, with the healthcare app used, index data can be presented graphically to the user from a variety of viewpoints. This can facilitate the user to recognize a tendency of the index data.

2.10 Combination of Panels

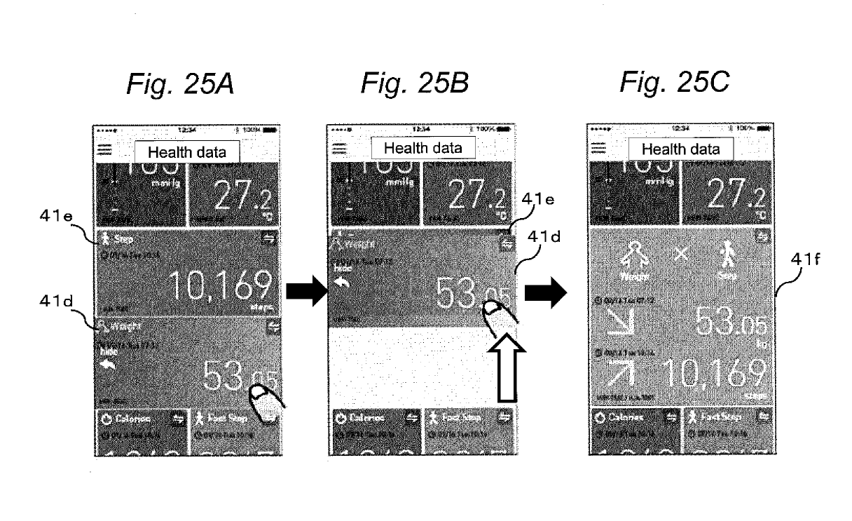

The healthcare app can combine a plurality of index panels in accordance with user's operation. For example, as sequentially shown in FIGS. 25A to 25C, by moving an index panel 41d for a weight (Weight) to be overlapped on an index panel 41e for the number of steps (Step), the index panel 41d for the weight (Weight) and the index panel 41e for the number of steps (Step) are combined to display a combined index panel 41f. In this case, the indexes of the weight and the number of steps are displayed on a single index panel 41f, enabling the user to realize the correlation between the data concerning a plurality of relevant indexes.

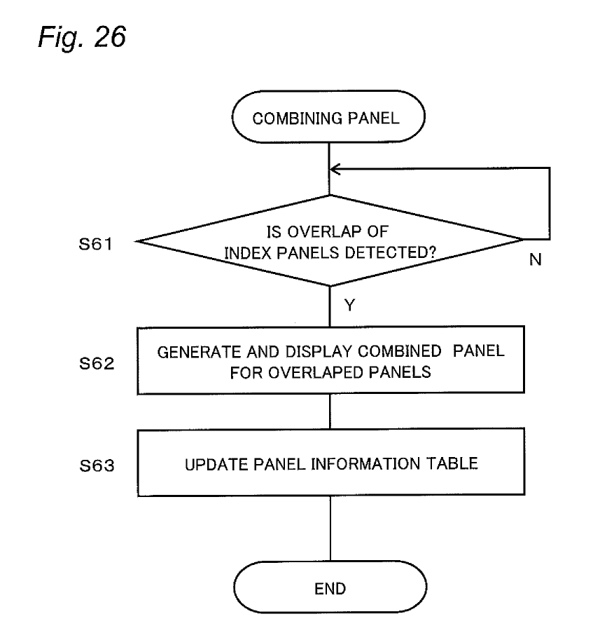

With reference to a flowchart of FIG. 26, the control of combining index panels will be described. Upon detection of overlap of the index panels (YES in S61), the controller 11 generates a combined panel for the overlapped index panels and displays the generated combined panel on the display unit 13 (S62). The controller 11 then updates the panel information table 33 (S63). Specifically, the controller 11 adds information about the newly generated combined panel to the panel information table 33 and updates information about the original index panels before combined.

The overlap of the index panels here means a state where at least most of the whole (a predetermined percentage or more) of one index panel is overlapped on the other index panel so that the most part of one index panel is included in the other index panel. In other words, when a rate of a non-overlapped region to the whole region of each index panel is not less than a predetermined value, the index panels are considered as not overlapped. In this case, the processing for moving the index panel is performed (see the processing as shown in FIG. 20).

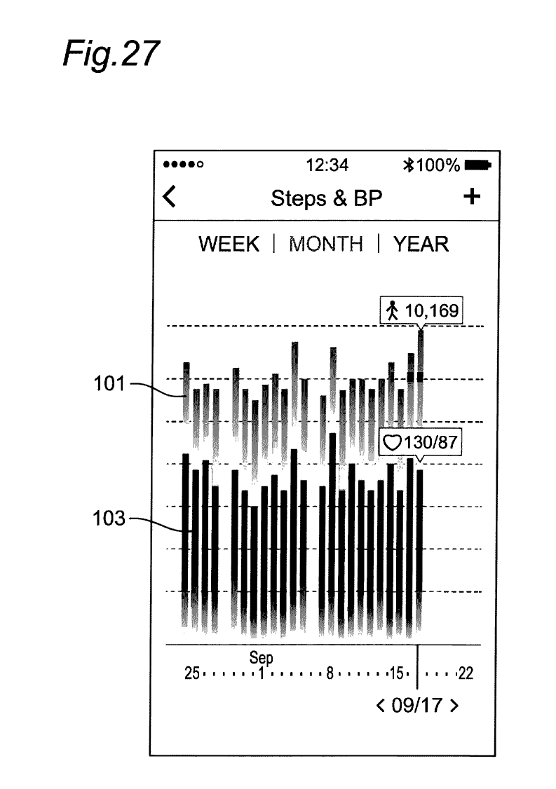

When a region on the combined panel is tapped by the user, charts respectively showing two indexes are combined and displayed as shown in FIG. 27. FIG. 27 shows an example in which a chart 101 concerning the number of steps and a chart 103 concerning a blood pressure are combined and displayed. Referring to such charts, the user can intuitively grasp the correlation between two indexes (the relation between the number of steps and the blood pressure in the example of FIG. 27).

2.11 Non-Display of Index Panel

The user can hide the index panel displayed on the home screen. This can simplify the display of the home screen so that interesting indexes can be viewed within the same field of view. For example, a delete button may be provided on the index panel, and when the user performs tap operation on the delete button, the controller 11 hides the index panel corresponding to the delete button. At this time, the controller 11 deletes information of the index panel corresponding to the delete button from the panel information table 33. Note that the index panel can be hidden also by moving the index panel to the outside of the screen region of the display unit 13 as described above.

2.12 Display of Index Panel Upon Cancellation of Registration of Measuring Device

When cancelling registration of a measuring device registered in the mobile data terminal 1 (the device information table 32), if any measuring device in the same category as the cancelled measuring device is not registered, the index panel of the measuring device of which registration is cancelled is not deleted. That is, even after cancellation of the registration of the measuring device, the index panel of the cancelled measuring device is kept being displayed as an index panel not linked with a measuring device. This allows the user to store and refer to past measurement results of that category. In this case, regarding the index panel corresponding to the cancelled measuring device, the controller 11 sets the device registration flag to “OFF” in the panel information table 33.

When a new measuring device of the same category as the category of the index panel corresponding to the cancelled measuring device is added while the index panel corresponding to the cancelled measuring device is displayed, the index panel corresponding to the cancelled measuring device is deleted and an index panel associated with the new measuring device is added. At this time, the controller 11 deletes from the panel information table 33 the information (the record) of the index panel corresponding to the cancelled measuring device, and adds information of the new index panel to the panel information table 33. Note that the past measurement results in the index database 34 are taken over by the newly added index panel. This allows the user to continue to hold and refer to his or her own measurement results.

### 3. Summary

As described above, in the health data management system 100 of the first embodiment, the mobile data terminal 1 (an example of the health data management device) includes the second wireless communication unit 19 (an example of the communication unit) that receives index data based on a measurement result from the measuring device 5 for measuring biological information (a body temperature, a blood pressure, and the like) and activity information (the number of steps, a walking distance, and the like) of the user (an example of the person to be measured) through wireless communication, the data storage unit 17 (an example of the storage unit) that stores the index database 34 for managing the received index data, the display unit 13 (an example of the display unit) that displays index data managed in the index database 34 in a predetermined display method, and the controller 11 (an example of the control unit) that controls an image displayed on the display unit 13. The controller 11 displays, on the display unit 13, an index panel that is an icon showing a value of each index data, for at least a part of index data managed in the index database 34.

As described above, the index database 34 is provided inside the mobile data terminal 1 to manage an index measured by each measuring device 5. Thus, for referring to data managed in the index database 34, connection with the server on the network is unnecessary, making the data in the index database 34 usable regardless of a network connection environment. Each of the index data managed in the index database 34 is displayed as the index panel on the display unit 13. At that time, the data is displayed in a format easily recognizable by the user (a changing panel color, a different panel size, a chart). By referring to this display, the user can easily grasp the state of each index. Data from each measuring device 5 can be transmitted to the mobile data terminal 1 by simple operation at the user's desired timing. Alternatively, data can be automatically transferred to the mobile data terminal 1 without making the user conscious of it. The latest information is thereby stored in the mobile data terminal 1, and the user can check the information. Hence, the health data management system 100 for the user of the first embodiment can improve the convenience of the user in terms of the healthcare.

Note that in the first embodiment, the touch panel and the button have been shown as examples of the operation units 15 and 55 in the mobile data terminal 1 and the measuring device 5. However, the operation unit is not restricted to these, and examples thereof may include any input means and any instruction means. Examples of the operation unit may include a touch pad, a switch, a keyboard, a mouse, and the like.

In the first embodiment, data is transferred between the mobile data terminal 1 and the measuring device 5 in accordance with the Bluetooth standard. However, the standard for communication between the mobile data terminal 1 and the measuring device 5 is not restricted to the Bluetooth. This standard may simply be a standard for communication between devices located relatively adjacent to each other. For example, communication may be performed in accordance with NFC (near field communication) or WiFi.

The control program (healthcare app) executed by the controllers 11 and 51 of the mobile data terminal 1 and the measuring device 5 may be provided by a recording medium such as a DVD-ROM or a CD-ROM, or may be downloaded from a server on a network through a communication circuit. In the first embodiment, the function of each controller 11 or 51 is realized by cooperation of the hardware and the software (application program). However, it may be realized only by a hardware circuit exclusively designed to realize a predetermined function. Hence the controller 11 can be configured by not only a CPU or an MPU, but also a DSP, an FPGA, an ASIC, or the like.

The type of the measuring device 5 and the types of biological information and action information measured by the measuring device 5 are not restricted to those described above.

### Present Disclosure

The above embodiment discloses the following device, system, and program.

A health data management device according to a first aspect of the present disclosure includes: a communication unit that receives index data based on a result of measurement from a measuring device for measuring biological information and activity information of a person to be measured through wireless communication; a storage unit that stores database for managing the received index data; a display unit that displays index data managed in the database in a predetermined display method; and a control unit that controls an image displayed on the display unit. The control unit causes an index panel of each index data to be displayed on the display unit for at least a part of the index data managed in the database, the index panel being an icon showing a value of each index data.

In this manner, the database is provided inside the health data management device, managing index data measured by the measuring device. Hence, in referring to data managed in the database, the health data management device does not need to be connected with a server over a network, so that data in the database can be used regardless of a network connection environment. Each of the index data managed in the database is displayed on the index panel on the display unit, and thus the user can visually and easily recognize the index data. For the above reason, the present health data management device enables easy use of index data showing a health condition of an individual which is measured by the measuring device.

Further, in the health data management device, the control unit may change a color of a background of the index panel in accordance with a period for which the index data shown by the index panel has not been updated. Referring to the index panel with color changed in this manner, the user can easily grasp an index panel with data which has not been updated for a long time.

The health data management device may further include an operation unit that receives user operation. When operation of pulling down a screen of the display unit is performed on the operation unit, the control unit may attempt to receive index data from a plurality of measuring devices previously registered through the communication unit. Accordingly, index data can be received (updated) in a batch from a plurality of measuring devices by simple user operation.

When the health data management device includes the operation unit and when predetermined operation is done on the index panel, the control unit may attempt to receive index data from a measuring device corresponding to the index panel through the communication unit. Hence the user can receive (update) index data for each measuring device.

In the health data management device, the control unit may execute a predetermined program to realize a predetermined function. At that time, when the predetermined program is executed in the back ground, the control unit may attempt to receive index data from a plurality of measuring devices previously registered through the communication unit every predetermined period. Hence index data can be automatically received (updated) without making the user conscious of it.

When the health data management device includes the operation unit, upon making a predetermined operation on the index panel, the control unit may display, on the display unit, a chart concerning index data shown by the index panel. With the graphical display, the user can more easily grasp a variation in index data.

When the health data management device includes the operation unit, upon making operation of overlapping two index panels, the control unit may display on the display unit one index panel showing two index data shown by the respective two index panels. By referring to such one index panel showing two index data, the user can easily grasp the correlation between two indexes.

Further in the health data management device, a size of one index panel showing index data that represents a measuring device in a plurality of index panels corresponding to the measuring device may be made larger than sizes of the other index panels. This can direct the user's attention to the most important index data in the measuring device.

The index data may include at least one of a body temperature, a blood pressure, a blood glucose level, a weight, a body fat, and the number of steps, but is not restricted to these values.

The health data management device may be a smartphone, for example.

A health data management system according to a second aspect of the present disclosure includes: a measuring device that measures biological information and activity information of a person to be measured, and transmits index data based on a result of the measurement; and the health data management device that receives index data from the measuring device.

Similarly to the health data management device according to the first aspect, this health data management system also allows the user to easily use index data which shows a health condition of an individual and is measured by the measuring device.

A recording medium according to a third aspect of the present disclosure records a program. The program is a program for making a computer of an information processor execute the functions of: receiving index data based on a result of measurement from a measuring device for measuring biological information and activity information of a person to be measured through wireless communication; registering the received index data on a database provided in the information processor; and displaying, on a display unit of the information processor, an index panel that is an icon showing a value of each index data, for at least a part of index data registered in the database.

With this program, a data management device enables the user to easily use index data which shows a health condition of an individual and is measured by a measuring device.

Although the present invention has been described in association with the above specific embodiment, a large number of modifications, collections, and applications are apparently made by those skilled in the art. Hence the present invention is not restricted to the above disclosure, but is restricted only by the attached claims.

What is claimed is:

1. A health data management device comprising:
a communication unit that receives index data based on a result of measurement from a measuring device for measuring biological information and activity information of a person to be measured, through wireless communication;
a storage unit that stores a database for managing the received index data;
a display unit that displays index data managed in the database, in a predetermined displaying method; and
a control unit that controls an image displayed on the display unit, wherein
the control unit causes an index panel of each index data to be displayed on the display unit, for at least a part of the index data managed in the database, the index panel being an icon showing a value of each index data.

2. The health data management device according to claim 1, wherein the control unit changes a color of a background of the index panel in accordance with a period for which the index data shown by the index panel has not been updated.

3. The health data management device according to claim 1, further comprising
an operation unit that receives user operation,
wherein, when an operation of pulling down a screen of the display unit is performed on the operation unit, the control unit attempts to receive index data from a plurality of measuring devices previously registered through the communication unit.

4. The health data management device according to claim 1, further comprising
an operation unit that receives an operation by a user,
wherein, when a predetermined operation is performed on the index panel, the control unit attempts to receive index data from a measuring device corresponding to the index panel through the communication unit.

5. The health data management device according to claim 1, wherein
the control unit executes a predetermined program to realize a predetermined function, and
when the predetermined program is executed in back ground, the control unit attempts to receive index data from a plurality of measuring devices previously registered, through the communication unit every predetermined period.

6. The health data management device according to claim 1, further comprising
an operation unit that receives user operation,
wherein, when a predetermined operation is performed on the index panel, the control unit displays on the display unit a chart concerning index data shown by the index panel.

7. The health data management device according to claim 1, further comprising
an operation unit that receives user operation,
wherein, when an operation of overlapping two index panels is performed, the control unit displays, on the display unit, one index panel showing two index data shown by the respective two index panels.

8. The health data management device according to claim 1, wherein a size of one index panel showing index data that represents a measuring device in a plurality of index panels corresponding to the measuring device is made larger than sizes of the other index panels.

9. The health data management device according to claim 1, wherein the index data includes at least one of a body temperature, a blood pressure, a blood glucose level, a weight, a body fat, and the number of steps.

10. The health data management device according to claim 1, which is a smartphone.

11. A health data management system, comprising:
a measuring device that measures biological information and activity information of a person to be measured, and transmits index data based on a result of the measurement; and
a health data management device according to claim 1 which receives index data from the measuring device.

12. A non-transitory recording medium storing a program making a computer of an information processor to execute the functions of:
receiving index data based on a result of measurement from a measuring device for measuring biological information and activity information of a person to be measured, through wireless communication;
registering the received index data on a database provided in the information processor; and
displaying, on a display unit of the information processor, an index panel that is an icon showing a value of each index data, for at least a part of the index data registered in the database.
